(19) United States
(12) Patent Application Publication
(10) Pub. No.: US 2004/0191339 A1
(43) Pub. Date: Sep. 30, 2004

(54) Cosmaceutical cream containing aqueous raspberry seed extract compounded to inhibit collagenase enzymes

(76) Inventor: Robert D. Bibb, Myrtle Beach, SC (US)

Correspondence Address: Robert D. Bibb, M.D., 7554 Siena Blvd, Myrtle Beach, SC, 29572, US

(21) Appl. No.: 10/393,656

(22) Filed: Mar. 24, 2003

(51) Int. Cl. A61K 35/78

(52) U.S. Cl. 424/765

(57) ABSTRACT

An epidermal cosmaceutical hypoallergenic cream containing aqueous raspberry seed extract of specific raspberry variants in concentrations ranging from 5 to 20% by weight for the express purpose of inhibiting collagenase enzymes 2 and 9.

### FIELD OF THE INVENTION

The present invention relates generally to an epidermal topical formulation containing aqueous extraction components of certain red raspberry seed variants compounded into a hypoallergenic cream base for treating the negative effects associated with exposure of the human skin to ultraviolet light. In particular, the cream relates to blockage of certain enzymes known as collagenases and more specifically to blockage of collagenases numbers two and nine.

### BACKGROUND OF THE INVENTION

Over the counter cosmaceutical creams have been in use for years and have been promoted to treat various maladies of the skin. Until recently the mechanisms behind the skin aging process have been elusive. Recent studies by Inomata et al led to the discovery of one very important process in the injury of the skin. ¹ One such mechanism involves the activation of collagenase enzymes by ultraviolet light B. In particular, ultraviolet light B is very effective in activating collagenases two and nine. The collagenase enzymes then injure the collagen structure of the skin, leading to wrinkling. ¹ Inomata S, et al. Possible involvement of gelatinases in basement membrane damage and wrinkle formation in chronically ultraviolet B-exposed hairless mouse. J Invest Dermatology 2003 January: 120(1):128-34

Recent research at Clemson University has determined that aqueous extracts of certain raspberry seed variants inhibit collagenase enzymes two and nine by almost 100% at very low concentrations. This inhibition constitutes the basis for the patent application. A cosmaceutical cream containing the aqueous raspberry seed extract has been developed. While other cosmaceutical creams for wrinkling have been developed none contains a natural material with collagenase enzyme two and nine inhibitory capabilities.

### SUMMARY OF THE INVENTION

According to its major aspects and briefly stated, the present invention is a new epidermal, hypoallergenic cosmaceutical cream containing aqueous extracts of certain raspberry seed varieties. The invention is designed to inhibit the activity of certain enzymes known as collagenases. In particular, the aqueous raspberry seed extract has been demonstrated to interfere with collagenase enzymes two and nine. These enzymes are known to participate in the aging process linked to wrinkling.

The present invention represents a significant departure from existing cosmaceutical creams in that it contains aqueous extracts from seed flour of specific red raspberry varieties.

A primary feature of the present invention is the aqueous extracts of certain red raspberry seed flour and the manner in which it is prepared. It is generally known that raspberries contain in excess of over 200 compounds. Many of these compounds are water-soluble and are contained within the seed, which is protected by the husk. The husk acts as a barrier to aqueous extraction of these compounds. Milling the seeds of Meeker and Chiliwak varieties into micronized flour overcomes this barrier. Once the flour has been created the extract is obtained by heating the raspberry seed flour in distilled water for a minimum of 20 minutes. The resultant supernatant is then distilled down to a concentrate. Raspberry seed flour is a feature of the present invention because the seed husks prevent any significant extraction of the water-soluble compounds.

The extract obtained from the heated water technique is then incorporated into a colloidal cream base designed to be applied to the skin with finger application. As it pertains to the present invention, quantified amounts of the aqueous extract in weight equivalents of 5% to 20% are incorporated into a hypoallergenic cream base. The resulting cream is applied to the skin in a twice-daily regimen.

### DETAILED DESCRIPTION OF PREFERRED EMBODIMENTS

The present invention is a new cosmaceutical cream containing aqueous extracts of Meeker and Chiliwak red raspberry seed flour incorporated into a colloidal cream base of hypoallergenic nature.

Raspberry seed flour is made by finely milling dried raspberry seeds of the varieties described above into micronized flour. Once the flour is obtained the extract is made by heating the seed flour in distilled water for a minimum of 20 minutes. The resultant supernatant is then distilled down to a concentrate, which is incorporated into a hypoallergenic cream base with the following composition:

| | |
|---|---|
| Purified Water | Tocopherol |
| Raspberry Seed Extract | Lactic Acid |
| C12-15 Alkyl Benzoate | Squalene |
| Sorbitol | Triticum Vulgare(Wheat) Protein |
| Glyceryl Stearate | Laureth-7 |
| Isopropyl Palmitate | Retinyl Palmitate |
| Ethoxydiglycol | Cetyl Alcohol |
| Dimethicone | Phenoxyethanol |
| Polyacrylamide | Tetrasodium EDTA |
| PEG-100 Stearate | Propylparaben |
| Beeswax | Xanthan Gum |
| Sorbitan Stearate | Ethyparaben |
| C13-14 Isoparaffin | Butylparaben |
| Fragrance | |

What is claimed is:

1. An epidermal topical formulation comprising raspberry seed flour extract.

2. The epidermal topical formulation as recited in claim 1, wherein said raspberry seed flour extract is from Meeker and Chiliwak raspberry varieties.

3. The epidermal topical formulations as recited in claim 1, wherein said raspberry seed flour is dried and micronized.

4. The epidermal topical formulation as recited in claim 1, wherein said raspberry seed flour extract is from an aqueous extraction process.

5. The epidermal topical formulation as recited in claim 1, wherein said formulation is a hypoallergenic cream.

6. The epidermal topical formulation as recited in claim 1 requires milling raspberry seeds into micronized flour and heating said flour in distilled water for a minimum of 20 minutes. The aqueous supernatant is separated from the heated seed flour.

7. The aqueous supernatant recited in claim 7, wherein said supernatant is incorporated into a cream base comprising 5-20% by weight.
